(19) United States
(12) Patent Application Publication
(10) Pub. No.: US 2010/0216817 A1
(43) Pub. Date: Aug. 26, 2010

(54) Antitumoral Tetrahydro-Pyrimidines

(75) Inventors: José Fernando Reyes Benitez, Madrid (ES); Maria del Carmen Cuevas Marchante, Madrid (ES); David Montalvo Lobo, Madrid (ES); Maria-Jesús Martin López, Madrid (ES); Maria Cristina Mateo Urbano, Madrid (ES); Andrés Francesch Solloso, Madrid (ES)

Correspondence Address: KING & SPALDING, 1185 AVENUE OF THE AMERICAS, NEW YORK, NY, 10036-4003, US

(73) Assignee: Pharma Mar SA, Madrid (ES)

(21) Appl. No.: 11/913,513

(22) PCT Filed: May 3, 2006
(86) PCT No.: PCT/EP2006/004117

§ 371 Date: Feb. 22, 2008

(30) Foreign Application Priority Data
May 3, 2005 (EP) 05076037.0

(51) Int. Cl. A61K 31/505; C07D 239/06; A61P 35/00

(52) U.S. Cl. 514/256; 544/242; 544/335

(57) ABSTRACT

Antitumoral compounds of the formula (I) wherein X is O, S, or NRa, obtained from a maze coral of the family Meandrinidae, genus Meandrina, species meandrites, or derivatives thereof are useful as antitumoral agents.

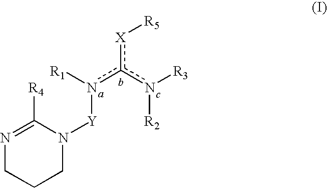

### FIELD OF THE INVENTION

The present invention relates to new antitumoral compounds, pharmaceutical compositions containing them and their use as antitumoral agents.

### BACKGROUND OF THE INVENTION

Kourany-Lefoll et al. (J. Org. Chem. 1992, 57, 3832-3835) disclosed the biological activities of Phloeodictine A (1) and Phloeodictine B (2), the first naturally occurring members of bicyclic 1,2,3,4-tetrahydro-6H-pyrrolo[1,2-α]pyrimidinium ring system, obtained from an undescribed species of the deep water sponge Phloeodictyon sp.

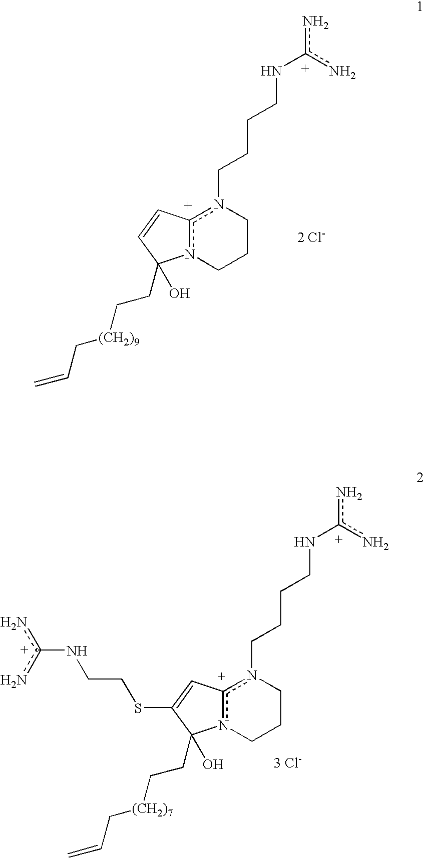

It is described that compounds 1 and 2 exhibited significant activity against several bacteria with the following respective MIC's (μg/mL): Streptococcus fecalis (5, >15), Staphylococcus aureus (1, 3), Escherichia coli (1, 30), and Pseudomonas aeruginosa (10, >30).

In addition, these alkaloids exhibited in vitro cytotoxicity against KB human nasopharyngeal carcinoma cells with IC₅₀ of 1.5 and 11.2 μg/mL for 1 and 2, respectively.

Also, Kourany-Lefoll et al. (Tetrahedron 1994, 50, 3415-3426) described later the pyrrolo[1,2-α]pyrimidines named Phloeodictines A1-A7 (3-9) and C1-C2 (10 and 11), isolated in further search for bioactive agents from the same sponge. All compounds exhibited in vitro antibacterial activities and were moderately cytotoxic against KB cells.

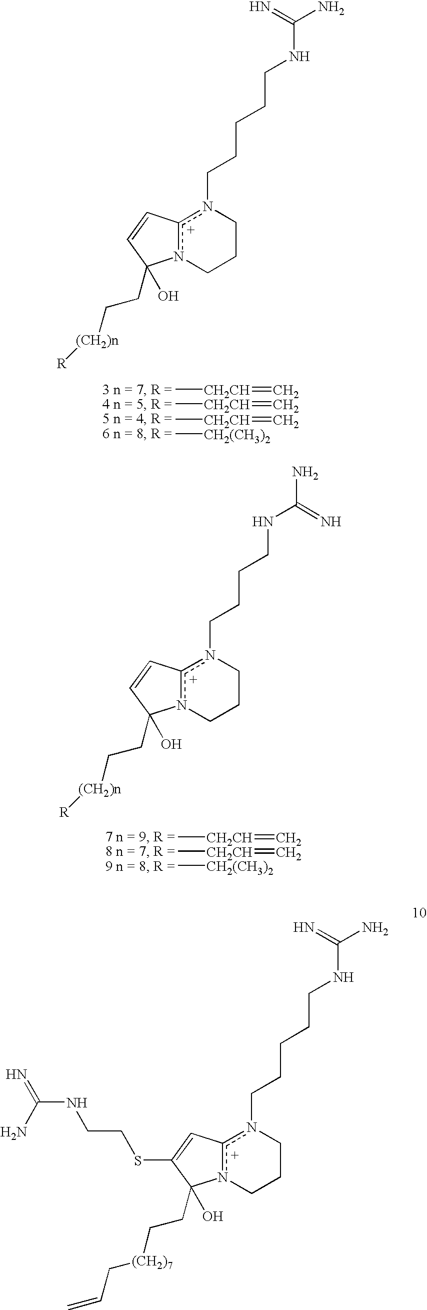

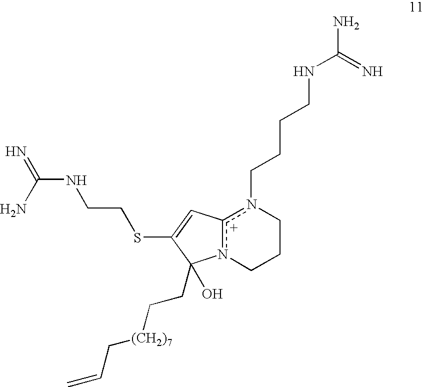

Mixtures of Phloeodictines A, B, A 1-A7 and C1-C2 have a wide spectrum of activity with the following respective MIC's (μg/mL): Staphylococcus aureus (3, 30, 1, 3), Escherichia coli (3, 30, 3, >30), Pseudomonas aeruginosa (30, >30, 30, >30), Clostridium perfringens (30, >30, 1, >100), Bacteroides fragilis (1, >30, 3, >100) and Peptococcus assaccharolyticus (10, >30, 3, >100). In addition, these substances also exhibit in vitro cytotoxicity towards KB human nasopharyngeal carcinoma cells with IC₅₀ of 2.2, 3.5, 0.6 and 1.8 μg/mL for Phloeodictine A, B, A 1-A7, and C1-C2, respectively.

U.S. Pat. No. 4,292,429 discloses activity against epidermoid carcinomas induced by diethylnitrosamine (DAENA) in the lungs, the trachea and the larynx in Syrian golden hamsters or against the Erhlich ascites carcinoma in mice of 1-[2-[2-(2-chlorophenyl)-2-imidazolin-1-yl]-ethyl]-3-(p-tolyl)-urea, 1-[2-[2-(4-pyridyl)-2-imidazolin-1-yl]-ethyl]-3-(-4-carboxy-phenyl)-urea and 1-[2-[2-(chloroanilinomethyl)-2-imidazolin-1-yl]-ethyl]-3-(p-tolyl)-urea.

On the other hand, some other 1,2-disubstituted cyclic amides have been disclosed in an unrelated area of the prior art. Specifically, U.S. Pat. No. 2,743,255 discloses a process for the preparation of resins which are valuable as chemical reactants. The following compound R is disclosed as a suitable reactant in the preparation of said resins:

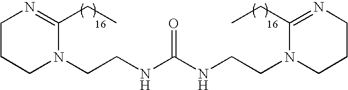

Cancer is a leading cause of death in animals and humans. Several efforts have been and are still being undertaken in order to obtain active and safe antitumor agents to be administered to patients suffering from a cancer. The problem to be solved by the present invention is to provide further compounds that are useful in the treatment of cancer.

### SUMMARY OF THE INVENTION

In one aspect, the present invention is directed to compounds of general formula I or a pharmaceutically acceptable salt, derivative, tautomer, prodrug or stereoisomer thereof,

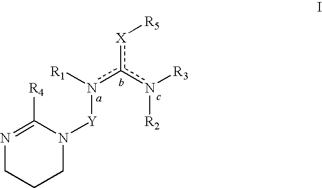

wherein R₁, R₂, R₃ and R₅ are each independently selected from the group consisting of H, OH, NO₂, NH₂, SH, CN, halogen, C(═O)H, CO₂H, COOalkyl substituted or unsubstituted C₁-C₁₂ alkyl, substituted or unsubstituted C₂-C₁₂ alkenyl, substituted or unsubstituted C₂-C₁₂ alkynyl, substituted or unsubstituted C₄-C₁₈ aryl, substituted or unsubstituted C₄-C₁₈ heterocyclic group, substituted or unsubstituted C₁-C₁₂ alkoxy and substituted or unsubstituted C₂-C₁₂ acyl;
Y is selected from the group consisting of substituted or unsubstituted C₁-C₁₂ alkylene, substituted or unsubstituted C₂-C₁₂ alkenylene and substituted or unsubstituted C₂-C₁₂ alkynylene;
X is selected from the group consisting of O, S and NRₐ;
Rₐ is selected from the group consisting of H, OH, NO₂, NH₂, SH, CN, halogen, C(═O)H, CO₂H, COOalkyl, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₄-C₁₈ aryl, substituted or unsubstituted C₄-C₁₈ heterocyclic group, substituted or unsubstituted C₁-C₁₂ alkoxy and substituted or unsubstituted C₂-C₁₂ acyl;
R₄ is selected from the group consisting of substituted or unsubstituted C₁-C₃₀ alkyl, substituted or unsubstituted C₂-C₃₀ alkenyl, substituted or unsubstituted C₂-C₃₀ alkynyl and substituted or unsubstituted C₄-C₃₀ alkenynyl; and
the dotted line represents an optionally additional bond which is placed in Nₐ—C_{b}, being R₁ absent, in C_{b}—X, being R₅ absent or in C_{b}—N_{c}, being R₂ absent;
with the exception of compound R of formula:

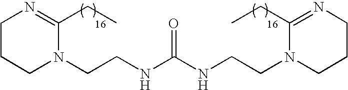

The present invention also relates to the isolation of the compounds of formula I from a maze coral of the family Meandrinidae, genus Meandrina, species meandrites, and the formation of derivatives from these compounds.

In another aspect, the present invention is also directed to the use of compounds of formula I including compound R, or pharmaceutically acceptable salts, tautomers, derivatives, prodrugs or stereoisomers thereof in the treatment of cancer, or in the preparation of a medicament for the treatment of cancer.

In another aspect, the present invention is also directed to pharmaceutical compositions comprising a compound of formula I including compound R, or pharmaceutically acceptable salts, tautomers, derivatives, prodrugs or stereoisomers thereof together with a pharmaceutically acceptable carrier or diluent.

### DETAILED DESCRIPTION OF PREFERRED EMBODIMENTS

The present invention relates to compounds of general formula I as defined above.

In these compounds the substituents can be selected in accordance with the following guidance:

Alkyl, alkylene and alkoxy groups preferably have from 1 to 30 carbon atoms. One more preferred class of alkyl, alkylene and alkoxy groups have from 1 to 12 carbon atoms, particularly preferred alkyl, alkylene and alkoxy groups have from 1 to about 6 carbon atoms, and most particularly preferred alkyl, alkylene and alkoxy groups have from 1 to about 4 carbon atoms. Methyl, ethyl, propyl including isopropyl and butyl are particularly preferred alkyl groups in the compounds of the present invention. Methoxy, ethoxy, propoxy including isopropoxy and butoxy including tert-butyl are particularly preferred alkoxy groups in the compounds of the present invention. Another more preferred class of alkyl and alkylene groups has from 4 to about 12 carbon atoms, yet more preferably from 5 to about 8 carbon atoms. Pentyl, hexyl, heptyl or octyl are particularly preferred alkyl groups in the compounds of the present invention. Another preferred class of alkyl group has from 1 to about 20 carbon atoms, yet more preferably from 6 to about 18 carbon atoms. As used herein, the term alkyl, unless otherwise modified, refers to both cyclic and noncyclic groups, although cyclic groups will comprise at least three carbon ring members.

We define alkenynyl group as an alkyl group containing one or more double bonds and one or more triple bonds, and preferred alkenynyl groups are those having from 4 to about 30 carbon atoms. One more preferred class of alkenynyl groups has from 6 to about 18 carbon atoms.

Preferred alkenyl, alkynyl, alkenylene and alkynylene groups in the compounds of the present invention have one or more unsaturated linkages and from 2 to about 30 carbon atoms. One more preferred class of alkenyl, alkynyl, alkenylene and alkynylene groups has from 4 to about 20 carbon atoms, and most preferably 6 to 18 carbon atoms. The terms alkenyl, alkynyl, alkenylene and alkynylene as used herein refer to both cyclic and noncyclic groups, although cyclic groups will comprise at least three carbon ring members. Another preferred class of alkenyl, alkynyl, alkenylene and alkynylene groups in the compounds of the present invention have from 2 to 12 carbon atoms, yet more preferably from 2 to 6 carbon atoms.

Alkyl, alkenyl, alkynyl, alkenynyl, alkylene, alkenylene and alkynylene groups may be optionally substituted by a group selected from OH, NO₂, SH, CN, halogen, C(═O)H, optionally substituted C₁-C₁₂ alkoxy, optionally substituted C₁-C₁₂ alkanoyloxy, optionally substituted C₄-C₁₉ aroyloxy, optionally substituted C₄-C₁₆ aralkanoyloxy, halogen, optionally substituted C₄-C₁₈ aryl, amino, mono-(C₁-C₁₂ alkyl)amino and di-(C₁-C₁₂ alkyl)amino, optionally substituted guanidine, optionally substituted C₁-C₁₂ alkoxycarbonyl, optionally substituted C₄-C₁₁ aryloxycarbonyl, optionally substituted C₄-C₁₁ aralkyloxycarbonyl, carbamoyl, N—(C₁-C₂₀ alkyl)carbamoyl and N,N-di-(C₁-C₂₀ alkyl)carbamoyl.

Suitable aryl groups in the compounds of the present invention include single and multiple ring groups, including multiple ring groups that contain separate or fused aryl or heteroaryl rings. Typical aryl groups contain from 1 to 3 rings and from 4 to about 18 carbon ring atoms. Specially preferred aryl groups include substituted or unsubstituted phenyl, naphthyl, biphenyl, phenanthryl and anthracyl.

Suitable heterocyclic groups include heteroaromatic and heteroalicyclic groups. Suitable heteroaromatic groups in the compounds of the present invention contain one, two or three heteroatoms selected from N, O and S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, imidazolyl, indolyl, benzofuranyl and benzothiazol groups. Suitable heteroalicyclic groups in the compounds of the present invention contain one, two or three heteroatoms selected from N, O and S atoms and include, e.g., tetrahydrofuranyl, tetrahydropyranyl, piperidinyl, morpholino and pyrrolidinyl groups.

Suitable acyl groups have from 2 to about 12 carbon atoms, more preferably from 2 to about 8 carbon atoms, still more preferably from 2 to about 6 carbon atoms, and even more preferably 2 carbon atoms.

Aryl, heterocyclic and acyl groups may be substituted at one or more available positions by one or more suitable groups such as OH, OR′, ═O, SH, SR′, SOR′, SO₂R′, NO₂, NH₂, NHR′, N(R′)₂, ═NR′, NHCOR′, N(COR′)₂, NHSO₂R′, NH(C═NH)NH₂, NH(C═NH)NHR′, NH(C═NH)NR′₂, CN, halogen, C(═O)H, C(═O)R′, CO₂H, CO₂R′, OC(═O)R′ wherein each of the R′ groups is independently selected from the group consisting of OH, NO₂, NH₂, SH, CN, halogen, ═O, C(═O)H, C(═O)CH₃, CO₂H, substituted or unsubstituted C₁-C₁₂ alkyl, substituted or unsubstituted C₂-C₁₂ alkenyl, substituted or unsubstituted C₂-C₁₂ alkynyl and substituted or unsubstituted aryl. Where such groups are themselves substituted, the substituents may be chosen from the foregoing list.

Suitable halogen substituents in the compounds of the present invention include F, Cl, Br and I.

The term “pharmaceutically acceptable salts, derivatives, prodrugs” refers to any pharmaceutically acceptable salt, ester, solvate, hydrate or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The term tautomer refers to one of two or more structural isomers of the defined compound, that exist in equilibrium and are readily converted from one isomeric form to another, such as amide-imide, lactam-lactim, etc.

The compounds of the invention may be in crystalline form either as free compounds or as solvates (e.g. hydrates) and it is intended that both forms are within the scope of the present invention. Methods of solvation are generally known within the art.

Any compound that is a prodrug of a compound of formula I is within the scope and spirit of the invention. The term “prodrug” is used in its broadest sense and encompasses those derivatives that are converted in vivo to the compounds of the invention. Such derivatives would readily occur to those skilled in the art, and include, for example, compounds where a free hydroxy group is converted into an ester derivative.

The compounds of the present invention represented by the above described formula I may include some type of enantiomers. Isomerism about the double bond is also possible, therefore in some cases the molecule could exist as (E)- or (Z)-isomer. The single isomers and mixtures of the isomers fall within the scope of the present invention.

Preferred compounds of the invention are those wherein Y is a substituted or unsubstituted C₁-C₆ alkylene, more preferably a substituted or unsubstituted C₁-C₄ alkylene. Methylene, ethylene, propylene, isopropylene and butylene are particularly preferred. Most preferred is an unsubstituted C₄ alkylene chain.

Particularly preferred R₁, R₂, R₃ and R₅ are each independently selected from the group consisting of H, OH, NO₂, NH₂, SH, CN, halogen, C(═O)H, CO₂H, COOalkyl, substituted or unsubstituted C₁-C₆ alkyl and substituted or unsubstituted C₂-C₆ acyl. In an embodiment they are all H.

In a preferred embodiment X is NRₐ, wherein Rₐ is preferably selected from the group consisting of H, OH, NO₂, NH₂, SH, CN, halogen, C(═O)H, CO₂H, COOalkyl, substituted or unsubstituted C₁-C₆ alkyl and substituted or unsubstituted C₂-C₆ acyl, being H and COOalkyl particularly preferred.

Particularly preferred compounds of the invention are those wherein R₄ is:

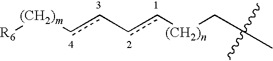

wherein n is an integer from 1 to 12, more preferred from 1 to 8;
m is an integer from 1 to 10 and particularly preferred from 1 to 5;
R₆ is selected from the group consisting of H, OH, NO₂, SH, CN, halogen, C(═O)H, optionally substituted C₁-C₁₂ alkoxy, optionally substituted C₁-C₁₂ alkanoyloxy, optionally substituted C₄-C₁₈ aroyloxy, optionally substituted C₄-C₁₆ aralkanoyloxy, optionally substituted C₄-C₁₈ aryl, amino, mono-(C₁-C₁₂ alkyl)amino, di-(C₁-C₁₂ alkyl)amino, optionally substituted guanidine, optionally substituted C₁-C₁₂ alkoxycarbonyl, optionally substituted C₄-C₁₁ aryloxycarbonyl, optionally substituted C₄-C₁₁ aralkyloxycarbonyl, carbamoyl, N—(C₁-C₂₀ alkyl)carbamoyl and N,N-di-(C₁-C₂₀ alkyl)carbamoyl; and
the dotted line represents an additional single or double bond. Particularly preferred is a double bond placed between C₁-C₂ and a triple bond placed between C₃-C₄.

Particularly preferred is the presence of an additional bond placed in Nₐ—C_{b}, being R₁ absent, in C_{b}—X, being R₅ absent or in C_{b}—N_{c}, being R₂ absent, and more preferably between C_{b} and X, being R₅ absent.

More particularly, the invention provides compounds of formula II:

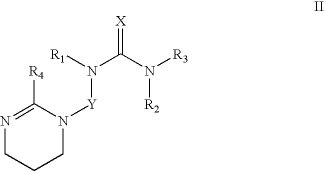

wherein R₁, R₂ and R₃ are each independently selected from the group consisting of H, OH, NO₂, NH₂, SH, CN, halogen, C(═O)H, CO₂H, COOalkyl, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₄-C₁₈ aryl, substituted or unsubstituted C₄-C₁₈ heterocyclic group, substituted or unsubstituted C₁-C₁₂ alkoxy and substituted or unsubstituted C₂-C₁₂ acyl;
Y is selected from the group consisting of substituted or unsubstituted C₁-C₁₂ alkylene, substituted or unsubstituted C₂-C₁₂ alkenylene and substituted or unsubstituted C₂-C₁₂ alkynylene;
X is selected from the group consisting of O, S and NH; and
R₄ is selected from the group consisting of substituted or unsubstituted C₁-C₃₀ alkyl, substituted or unsubstituted C₂-C₃₀ alkenyl, substituted or unsubstituted C₂-C₃₀ alkynyl and substituted or unsubstituted C₄-C₃₀ alkenynyl;
or a pharmaceutically acceptable salt, tautomer, derivative, prodrug or stereoisomer thereof.

Preferred compounds of formula II are those wherein Y is a substituted or unsubstituted C₁-C₆ alkylene, more preferably a substituted or unsubstituted C₁-C₄ alkylene. Methylene, ethylene, propylene, isopropylene and butylene are particularly preferred. Most preferred is an unsubstituted C₄ alkylene chain.

Particularly preferred R₁, R₂ and R₃ are each independently selected from the group consisting of H, OH, NO₂, NH₂, SH, CN, halogen, C(═O)H, CO₂H, COOalkyl, substituted or unsubstituted C₁-C₆ alkyl and substituted or unsubstituted C₂-C₆ acyl. In an embodiment they are all H.

In a preferred embodiment X is NH.

Particularly preferred compounds of the invention are those wherein R₄ is:

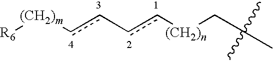

wherein n is an integer from 1 to 12, more preferred from 1 to 8;
m is an integer from 1 to 10 and particularly preferred from 1 to 5;
R₆ is selected from the group consisting of H, OH, NO₂, SH, CN, halogen, C(═O)H, optionally substituted C₁-C₁₂ alkoxy, optionally substituted C₁-C₁₂ alkanoyloxy, optionally substituted C₄-C₁₈ aroyloxy, optionally substituted C₄-C₁₆ aralkanoyloxy, optionally substituted C₄-C₁₈ aryl, amino, mono-(C₁-C₁₂ alkyl)amino, di-(C₁-C₁₂ alkyl)amino, optionally substituted guanidine, optionally substituted C₁-C₁₂ alkoxycarbonyl, optionally substituted C₄-C₁₁ aryloxycarbonyl, optionally substituted C₄-C₁₁ aralkyloxycarbonyl, carbamoyl, N—(C₁-C₂₀ alkyl)carbamoyl and N,N-di-(C₁-C₂₀ alkyl)carbamoyl; and
the dotted line represents an additional single or double bond. Particularly preferred is a double bond placed between C₁-C₂ and a triple bond placed between C₃-C₄.

Particularly preferred compounds of the invention are the following:

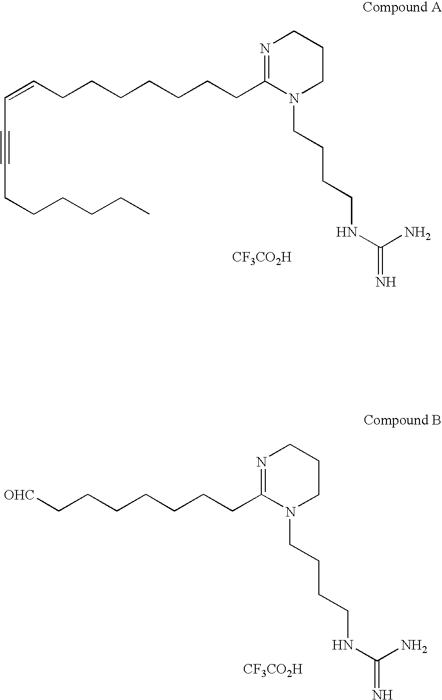

Compound A is a marine natural product isolated from a small sample of maze coral of the family Meandrinidae, genus Meandrina, species meandrites 31712. This coral was collected by scuba diving at the Caribbean Sea, near Motagua, at a depth of 40 m [UTM/NAD 1927 (North American Datum 1927, Zones 15 and 16) X Coordinate: 362642; Y Coordinate: 1751928], and its description is the following: The colonies are massive structures with meandroid or flabelloid forms and with polyps in the calcareous skeleton. The size can reach 30 cm in diameter with a pale yellow or brown colour.

Additionally, Compound A was synthesised following the synthetic process of Scheme 1.

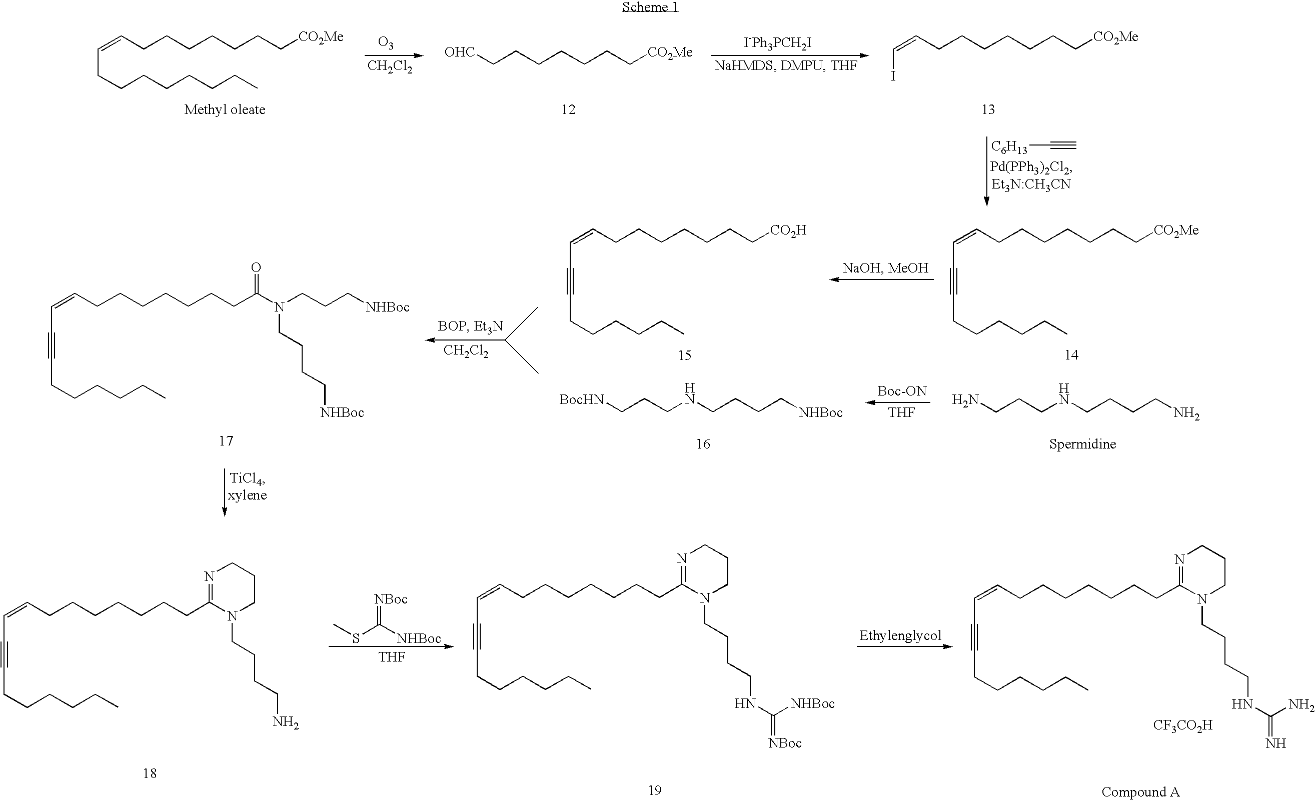

This process comprises the following sequential key steps:

a) Methyl oleate was subjected to oxidative cleavage of the carbon-carbon double bond to obtain the corresponding aldehyde 12;

b) aldehyde 12 was converted into the vinyl iodide 13 following standard literature procedures;

c) Sonogashira coupling reaction between the iodoalkenyl 13 and 1-octyne followed by hydrolysis of the ester group of enyne 14 in basic medium yielded acid 15;

d) coupling reaction between acid 15 and diprotected spermidine derivative 16 under standard literature conditions afforded the corresponding amide 17;

e) cyclization of 17 in the presence of TiCl₄ afforded the 1,4,5,6-tetrahydropyrimidine derivative 18; and

f) coupling reaction of 18 with N,N′-bis(tert-butoxycarbonyl)-2-methyl-2-thiopseudourea, followed by deprotection of the Boc groups of 19 yielded Compound A.

Analogues with different functional groups or substituents can be synthesized from this compound by usual procedures in synthetic organic chemistry and already known by a person skilled in the art. For example by hydrolysis, ozonolysis, Sharpless epoxidation or Diels-Alder reaction. In addition, analogues can also be synthesized using the procedures disclosed in scheme 1 with the appropriate intermediates.

An important feature of the above described compounds of formula I and II is their bioactivity and in particular their cytotoxic activity. With this invention we provide novel pharmaceutical compositions of compounds of general formula I and II that possess cytotoxic activity, and their use as antitumor agents. Thus the present invention further provides pharmaceutical compositions comprising a compound of this invention, a pharmaceutically acceptable salts, derivatives, prodrugs or stereoisomers thereof with a pharmaceutically acceptable carrier.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 2-12 hours, with 2-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may for example be delivered by liposome or nanosphere encapsulation, in sustained release formulations or by other standard delivery means.

The correct dosage of the compounds will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The compounds and compositions of this invention may be used with other drugs to provide a combination therapy. The other drugs may form part of the same composition, or be provided as a separate composition for administration at the same time or at different time.

Antitumoral activities of these compounds include leukaemia, lung cancer, colon cancer, kidney cancer, prostate cancer, ovarian cancer, breast cancer, pancreas cancer, cervix cancer, sarcomas and melanomas.

### EXAMPLES

### Example 1

### Description of the Coral and Collection Side

Samples of the maze coral of the family Meandrinidae, genus Meandrina, species meandrites 31712 were collected by scuba diving at Caribbean Sea, near Motagua at a depth of 40 m [UTM/NAD 1927 (North American Datum 1927, Zones 15 and 16) X Coordinate: 362642; Y Coordinate: 1751928].

### Example 2

### Isolation of Compound A

The frozen specimen (1646 g) of Example 1 was triturated and exhaustively extracted twice with isopropanol. The combined extracts were concentrated to yield a crude of 8.67 g. This material was resuspended in H₂O (500 mL) and extracted with Hexane (3×500 mL, 1.18 g yield), EtOAc (3×500 mL, 87 mg yield), and n-BuOH (2×250 mL, 394 mg yield).

Compound A (1.2 mg) was isolated from the active n-BuOH fraction by repeated semipreparative HPLC (SymmetryPrep C-18 7 μm, 7.8×150 mm column, H₂O (0.05% TFA):CH₃CN (0.05% TFA) gradient, UV detection).

Compound A: pale yellow oil. HRFABMS m/z 430.3917 [M+H]⁺ (calc. for C₂₆H₄₈N₅ 430.3910). ¹H (500 MHz) and ¹³C NMR (125 MHz) see Table 1.

TABLE 1
¹H and ¹³C NMR data of Compound A (CDCl₃).

| N^{o} | ¹³C δ | ¹H δ [m, J (Hz)] | COSY | gHMBC |
|---|---|---|---|---|
| 1 | 158.7 | — | — | H-2 |
| 2 | 42.0 | 3.22 (t, 7.0) | H-3 | H-3, H-4 |
| 3 | 26.8 | 1.63 (m) | H-2, H-4 | H-2, H-4, H-5 |
| 4 | 25.9 | 1.77 (m) | H-3, H-5 | H-2, H-3, H-5 |
| 5 | 52.4 | 3.50 (m) | H-4 | H-3, H-4, H-6 |
| 6 | 47.1 | 3.50 (m) | H-7, H-8 | H-5, H-7, H-8 |
| 7 | 19.9 | 2.02 (q, 6.0) | H-6, H-8 | H-6, H-8 |
| 8 | 39.9 | 3.36 (t, 6.0) | H-6, H-7 | H-6, H-7 |
| 9 | 165.0 | — | — | H-5, H-6, H-8, H-10, |
| | | | | H-11 |
| 10 | 32.4 | 2.54 (t, 8.0) | H-11, H-12 | H-11 |
| 11 | 28.0 | 1.63 (m) | H-10 | H-10 |
| 12 | 30.2* | 1.40 (m) | H-11 | H-10, H-11 |
| 13 | 30.1* | 1.40 (m) | — | — |
| 14 | 30.1* | 1.40 (m) | — | — |
| 15 | 30.0 | 1.40 (m) | — | H-16, H-17 |
| 16 | 31.0 | 2.27 (m) | H-15, H-17 | H-17, H-18 |
| 17 | 142.8 | 5.79 (dt, 10.5, 7.5) | H-16, H-18 | H-15, H-16 |
| 18 | 110.8 | 5.41 (d, 10.5) | H-17, H-21 | H-16 |
| 19 | 78.4 | — | — | H-17, H-21 |
| 20 | 110.8 | — | — | H-18, H-21, H-22 |
| 21 | 20.1 | 2.31 (dt, 6.5, 2.0) | H-18, H-22 | H-22 |
| 22 | 30.0 | 1.50 (m) | H-21, H-23 | H-21, H-23 |
| 23 | 29.6 | 1.40 (m) | H-22 | H-22 |
| 24 | 32.5 | 1.32 (m) | H-25 | H-22, H-23, H-25, H-26 |
| 25 | 23.7 | 1.32 (m) | H-24, H-26 | H-23, H-24, H-26 |
| 26 | 14.4 | 0.91 (t, 7.0) | H-25 | H-25 |

*Assignments may be interchanged

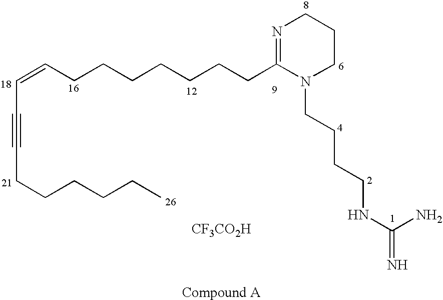

### Example 3

### Synthesis of Compound B

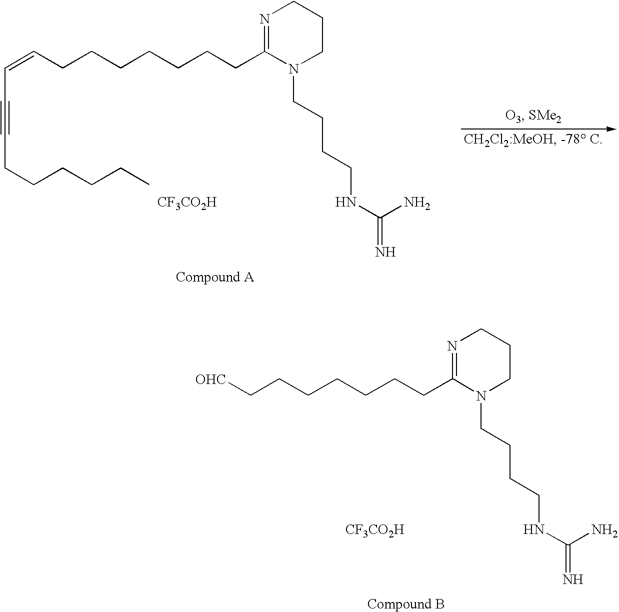

A stream of O₃ was bubbled through a solution of Compound A (8.0 mg, 0.015 mmol), in CH₂Cl₂:MeOH (1.0 mL:0.1 mL) at −78° C. until the mixture became blue. After bubbling a stream of Argon through the reaction at −78° C. during 10 min, dimethylsulfide (14 μL, 0.19 mmol) was added. The reaction was stirred at 23° C. during 30 min, and then the solvent was evaporated under vacuum to give a residue which was purified by HPLC (SymmetryPrep C-18 7 μm, 7.8×150 mm column, H₂O (0.1% TFA):CH₃CN (0.1% TFA) gradient, UV detection) to afford Compound B (2.8 mg, 46%).

¹H NMR (500 MHz, CD₃OD) δ 3.51 (m, 4H), 3.38 (t, J=6.0 Hz, 2H), 2.55 (t, J=7.8 Hz, 2H), 2.04 (m, 2H), 1.75 (m, 2H), 1.63 (m, 6H), 1.38 (m, 14H).

¹³C NMR (125 MHz, CD₃OD) δ 165.1, 162.9, 52.4, 47.1, 42.0, 39.9, 32.4, 30.8, 30.5, 30.2, 30.0, 29.9, 27.9, 26.8, 25.9, 19.9.

HRMS (MALDI): 324.2757 [M+H]⁺ (calculated for C₁₇H₃₄N₅O₁, 324.2763).

### Example 4

### Synthesis of Compound A

A solution of methyl oleate (10.0 g, 33.7 mmol) in anhydrous CH₂Cl₂ (100 mL) was cooled to −78° C. and a stream of O₃ was bubbled through the reaction mixture until the solution became lightly blue (10 min). Argon was bubbled through the mixture and a solution of PPh₃ (19.7 g, 75.1 mmol) in CH₂Cl₂ (100 mL) was added slowly. The reaction mixture was warmed to 23° C. and stirred for 18 hours. The solvent was evaporated to dryness and the solid was triturated with cold hexane (80 mL). The filtrated was evaporated to give a yellow oil. The oil was purified by chromatography on silica gel (CH₂Cl₂:Hex, 1:1 and then CH₂Cl₂:Et₂O, 1:1) to provide the two expected aldehydes, nonanal (4.80 g, 100%) and methyl 8-formyloctanoate 12 (6.28 g, 100%), both as colourless oils.

¹H NMR (300 MHz, CDCl₃) δ 9.76 (s, 1H), 3.66 (s, 3H), 2.41 (t, J=7.3 Hz, 2H), 2.30 (t, J=7.3 Hz, 2H), 1.61 (m, 4H), 1.31 (m, 6H).

¹³C NMR (75 MHz, CDCl₃) δ 202.4, 173.8, 51.1, 43.5, 33.7, 28.7, 28.6, 28.5, 24.5, 21.7.

MS (APCI): 187 (M+1)⁺. Rf=0.2 (CH₂Cl₂).

To a solution of (iodomethyl)triphenylphosphonium iodide (39.89 g, 75.3 mmol) in anhydrous THF (300 mL) NaHMDS (75.3 mL, 1.0M in THF, 75.3 mmol) was dropwise added and stirred for 10 min at 23° C. The reaction mixture was cooled to −60° C. and 1,3-dimethyl-3,4,5,6-tetrahydro-2(1H)-pyrimidinone (15.3 mL, 126.4 mmol) was dropwise added and immediately cooled to −78° C. A solution of methyl 8-formyloctanoate 12 (5.6 g, 30.1 mmol) in THF (290 mL) was slowly added to the ylide solution, over 30 min, stirred for 5 min at −78° C. and warmed to 23° C. After 2 hours, the mixture was diluted with hexane (300 mL) and washed with a saturated aqueous solution of NaCl (300 mL). The aqueous layer was extracted with hexane (3×300 mL) and the combined organic layers were dried over Na₂SO₄, filtered, and evaporated. Flash chromatography on silica gel (CH₂Cl₂:Hexane, 1:1) provided (Z)-methyl 10-iododec-9-enoate 13 (7.21 g, 77%) as a colourless oil.

¹H NMR (300 MHz, CDCl₃) δ 6.16-6.12 (m, 2H), 3.66 (s, 3H), 2.30 (t, J=7.3 Hz, 2H), 2.13 (m, 2H), 1.60 (m, 2H), 1.40-1.20 (m, 8H).

¹³C NMR (75 MHz, CDCl₃) δ 174.3, 141.3, 82.2, 51.4, 34.6, 34.0, 29.7, 29.0, 28.8, 27.8, 24.9.

MS (APCI): 184 (M−128)⁺. Rf=0.25 (CH₂Cl₂:Hexane, 1:1).

To a suspension of (Z)-methyl 10-iododec-9-enoate 13 (7.21 g, 22.9 mmol), Pd(PPh₃)₂Cl₂ (1.56 g, 2.29 mmol), and CuI (1.31 g, 6.88 mmol) in anhydrous acetonitrile:Et₃N (170 mL:34 mL) was added a solution of 1-octyne (4.06 mL, 27.51 mmol) in acetonitrile:Et₃N (50 mL:10 mL) over 4 hours at −20° C. The reaction mixture was warmed to 23° C. After 18 hours, HCl 1N (200 mL) was added and the mixture was extracted with CH₂Cl₂ (3×250 mL). The combined organic layers were dried over Na₂SO₄, filtered, and evaporated. Flash chromatography on silica gel (CH₂Cl₂:Hexane, from 10:1 to 1:1) provided (Z)-methyl octadec-9-en-11-ynoate 14 (5.41 g, 81%) as a colourless oil.

¹H NMR (300 MHz, CDCl₃) δ 5.80 (dt, J=10.3 and 7.3 Hz, 1H), 5.43 (d, J=10.3 Hz, 1H), 3.66 (s, 3H), 2.35-2.23 (m, 6H), 1.61-1.40 (m, 6H), 1.40-1.25 (m, 12H), 0.89 (br t, 3H).

¹³C NMR (75 MHz, CDCl₃) δ 174.3, 142.4, 109.4, 94.5, 77.3, 51.4, 34.1, 31.3, 29.9, 29.1, 29.0, 28.9, 28.8, 28.7, 28.5, 24.9, 22.5, 19.5, 14.0.

MS (APCI): 293 (M+1)⁺. Rf=0.30 (CH₂Cl₂:Hexane, 1:1).

To a solution of (Z)-methyl octadec-9-en-11-ynoate 14 (2.26 g, 8.12 mmol) in methanol (8.5 mL) was added a solution of 10M NaOH (1.62 mL, 16.2 mmol) at 23° C. The solution was stirred for 3 hours, then additional 10M NaOH (1.62 mL, 16.2 mmol) was added and 2 hours later a new addition of 10M NaOH (1.62 mL, 16.2 mmol) was done. 2 hours after the latest addition the reaction was completed and the solvent was evaporated under vacuum. The residue was diluted with H₂O and acidified with 1N HCl until pH=2. The aqueous layer was extracted with CH₂Cl₂ (2×200 mL), the combined organic layers were dried over anhydrous Na₂SO₄, filtered, and evaporated to dryness to give (Z)-octadec-9-en-11-ynoic acid 15 (2.0 g, 89%) as a colourless oil which was used without further purification.

¹H NMR (300 MHz, CDCl₃) δ 5.79 (dt, J=10.6 and 7.3 Hz, 1H), 5.42 (br d, J=10.6 Hz, 1H), 2.37-2.24 (m, 6H), 1.65-1.40 (m, 6H), 1.40-1.23 (m, 12H), 0.88 (t, J=7.0 Hz, 3H).

¹³C NMR (75 MHz, CDCl₃) δ 179.9, 142.4, 109.4, 94.5, 77.3, 34.0, 31.3, 29.9, 29.0, 28.9 (2), 28.8, 28.7, 28.5, 24.6, 22.6, 19.5, 14.0.

MS (APCI): 279 (M+1)⁺. Rf=0.40 (CH₂Cl₂:MeOH, 10:1).

To a solution of spermidine (3.0 g, 20.6 mmol) in THF (100 mL) was slowly added a solution of 2-(Boc-oxyimino)-2-phenylacetonitrile (10.14 g, 20.6 mmol) in THF (20 mL) at 0° C. The reaction mixture was stirred at 0° C. for 1 hour and then the solvent was evaporated under vacuum. The residue was filtrated on silica gel and eluted with CH₂Cl₂:EtOAc 7:3 and then with CH₂Cl₂:MeOH 1:1 to afford tert-butyl 4-(3-(tert-butyl carbamate)propyl)butylcarbamate 16 (5.3 g, >100%) as a colourless oil.

¹H NMR (300 MHz, CD₃OD) δ 3.15 (t, J=6.8 Hz, 2H), 3.08 (t, J=6.8 Hz, 2H), 3.00 (t, J=6.8 Hz, 4H), 1.83 (m, 2H), 1.69 (m, 2H), 1.55 (m, 2H), 1.44 (bs, 18H).

MS (APCI): 346 (M+1)⁺. Rf=0.18 (CH₂Cl₂:MeOH, 8:2).

To a solution of 15 (1.01 g, 3.65 mmol), and 16 (1.89 g, 5.47 mmol) in CH₂Cl₂ (50 mL) was added Et₃N (2.58 mL, 18.6 mmol) and Benzotriazol-1-yloxy)tris(dimethylamino)phosphonium hexafluorophosphate (2.42 g, 5.47 mmol) at 23° C. The reaction mixture was stirred for 3 hours at 23° C., then diluted with CH₂Cl₂, and washed with 1M HCl and a saturated aqueous solution of NaCl. The combined organic layers were dried over Na₂SO₄, filtered, and evaporated. The residue obtained was purified by flash chromatography on silica gel (CH₂Cl₂:EtOAc from 6:1 to 3:1) to afford compound 17 (2.14 g, 96%) as a colourless oil.

¹H NMR (500 MHz, CD₃OD) δ 5.79 (dt, J=10.5 and 7.5 Hz, 1H), 5.40 (bd, J=9.5 Hz, 1H), 3.34 (m, 4H), 3.07-3.01 (m, 4H), 3.35-2.25 (m, 4H), 1.76-1.31 (m, 16H), 1.43 (s, 28H), 0.90 (t, J=6.8 Hz, 3H).

¹³C NMR (125 MHz, CD₃OD) δ 175.5, 175.2, 158, 142.9, 110.7, 95.0, 78.4, 32.430.9, 30.5, 30.4, 30.3, 30.1, 29.9, 29.9, 29.5, 28.8, 23.6, 20.1, 14.4.

MS (APCI): 628 (M+23)⁺.

To a suspension of 17 (542 mg, 0.89 mmol) in anhydrous xylene (16 mL), TiCl₄ (98 μL, 0.89 mmol) was added slowly at 23° C. The mixture was heated at 165° C. for 1 hour. After cooled the reaction mixture to 23° C., a solution of NaOH (270 mg, 6.75 mmol) in MeOH (15 mL) was added, filtered through Celite® and washed with MeOH (20 mL). The filtrate was concentrated to dryness, a saturated aqueous solution of NaCl (50 mL) was added and the mixture was extracted with CH₂Cl₂ (3×50 mL). The combined organic layers were dried over anhydrous Na₂SO₄, filtered, and concentrated. The residue was purified by chromatography on silica-NH₂ gel (CH₂Cl₂:MeOH, from 16:1 to 1:1) to give (Z)-4-(2-(pentadec-6-en-8-ynyl)-5,6-dihydropyrimidin-1(4H)-yl)butan-1-amine 18 (131 mg, 38%) as a yellow oil.

¹H NMR (300 MHz, CD₃OD) δ 5.80 (dt, J=10.5 and 7.6, 1H), 5.41 (d, J=10.3 Hz, 1H), 3.49 (m, 4H), 3.37 (m, 2H), 2.71 (t, J=7.0 Hz, 2H), 2.54 (t, J=7.8 Hz, 2H), 2.32 (m, 4H), 2.02 (m, 2H) 1.69-1.30 (m, 22H) 0.92 (t, J=6.8 Hz, 3H).

¹³C NMR (75 MHz, CDCl₃) δ 163.1, 142.0, 109.2, 94.3, 76.9, 51.2, 45.7, 40.9, 38.5, 31.1, 29.7, 29.4, 28.8, 28.7, 28.5, 28.3, 27.3, 27.2, 25.2, 22.3, 19.3, 19.0, 13.8. (two ¹³C signals were not observed).

MS (APCI): 388 (M+1)⁺. Rf=0.32 (Si—NH₂, CH₂Cl₂:MeOH, 8:1).

To a solution of 18 (18 mg, 0.046 mmol) in anhydrous THF (0.6 mL) was added 1,3-bis(tert-butoxycarbonyl)-2-methyl-2-thiopseudourea (20 mg, 0.069 mmol) at 23° C. The reaction mixture was heated at 65° C. for 5 hours, then cooled to 23° C. Evaporation under vacuum gave a residue which was purified by chromatography on silica gel (CH₂Cl₂:MeOH from 99:1 to 9:1) to afford 19 (7.8 mg, 28%) as a colourless oil.

¹H NMR (300 MHz, CD₃OD) δ 5.79 (dt, J=11.0 and 7.5, 1H), 5.41 (m, 1H), 3.52 (m, 4H), 3.41 (t, J=6.5 Hz, 2H), 3.37 (t, J=6.0 Hz, 2H), 2.55 (t, J=7.5 Hz, 2H), 2.31 (td, J=7.0 and 2.0, 2H), 2.27 (t, J=7.0 Hz, 2H), 2.02 (m, 2H), 1.72 (m, 2H), 1.61 (m, 4H), 1.52 (s, 9H), 1.46 (s, 9H), 1.49-1.28 (m, 8H), 0.91 (t, J=7.0 Hz, 3H).

¹³C NMR (75 MHz, CD₃OD) δ 165.0, 164.5, 157.7, 154.2, 142.8, 110.8, 95.1, 84.5, 80.4, 78.4, 52.5, 47.1, 40.9, 39.9, 32.5, 32.4, 30.9, 30.1, 30.0, 29.59, 28.6, 28.55, 27.9, 27.1, 25.8, 23.7, 20.1, 20.0, 14.4.

MS (APCI): 630 (M+1)⁺. Rf=0.10 (CH₂Cl₂:MeOH, 94:6).

A solution of 19 (22 mg, 3.65 mmol) in ethylene glycol (2.2 mL) was heated at 200° C. for 2 min. The reaction mixture was cooled to 23° C. and partitioned into CH₂Cl₂ and a saturated aqueous solution of NaCl with drops of 3M NaOH (pH 14). The aqueous organic layer was extracted with CH₂Cl₂, and the combined organic layers were dried over Na₂SO₄, filtered, and evaporated under vacuum to give 31 mg of Compound A crude which was purified by HPLC (SymmetryPrep C-18 7 μm, 7.8×150 mm column, H₂O (0.1% TFA):CH₃CN (0.1% TFA) gradient, UV detection) to obtain Compound A (6.1 mg, 32%), which was identical in all parameters to those obtained in Example 2.

### Example 5

### Bioassays for Antitumor Screening

The finality of these assays is to interrupt the growth of a “in vitro” tumor cell culture by means of a continued exhibition of the cells to the sample to be testing.

CELL LINES

| | N° | | | |
|---|---|---|---|---|
| Name | ATCC | Species | Tissue | Characteristics |
| A549 | CCL- | human | lung | lung carcinoma |
| | 185 | | | “NSCL” |
| SK-MEL- | HTB-72 | human | melanoma | malignant melanoma |
| 28 | | | | |
| HT29 | HTB-38 | human | colon | colon adenocarcinoma |
| LoVo | CCL- | human | colon | colon adenocarcinoma |
| | 229 | | | |
| LoVo- | | human | colon | colon adenocarcinoma |
| Dox | | | | (MDR) |
| DU-145 | HTB-81 | human | prostate | prostate carcinoma, not |
| | | | | androgen receptors |
| LN-caP | CRL- | human | prostate | prostate adenocarcinoma, |
| | 1740 | | | with androgen receptors |
| SK-BR3 | HTB-30 | human | breast | breast adenocarcinoma, |
| | | | | Her2/neu+ (pleural |
| | | | | effusion) |
| IGROV | | human | ovary | ovary adenocarcinoma |
| IGROV- | | human | ovary | ovary adenocarcinoma, |
| ET | | | | characterized as ET- |
| | | | | 743 resistant cells |
| HeLa | CCL-2 | human | cervix | cervix epitheloid carcinoma |
| HeLa- | CCL-3 | human | cervix | cervix epitheloid carcinoma, |
| APL | | | | characterized as Aplidine |
| | | | | resistant cells |
| PANC1 | CRL- | human | pancreas | pancreatic epitheloid |
| | 1469 | | | carcinoma |
| MDA- | HTB-26 | human | breast | breast adenocarcinoma |
| MB-231 | | | | |

### Inhibition of Cells Growth by Colorimetric Assay

A colorimetric type of assay, using sulforhodamine B (SRB) reaction has been adapted for a quantitative measurement of cell growth and viability [following the technique described by Philip Skehan et al. (1990), New colorimetric cytotoxicity assay for anticancer drug screening, J. Natl. Cancer Inst. 82:1107-1112].

This form of assay employs 96 well cell culture microplates of 9 mm diameter (Mosmann, 1983; Faircloth, 1988). Most of the cell lines are obtained from American Type Culture Collection (ATCC) derived from different human cancer types.

Cells are maintained in RPMI 1640 10% FBS, supplemented with 0.1 g/L penicillin and 0.1 g/L streptomycin sulfate and then incubated at 37° C., 5% CO₂ and 98% humidity. For the experiments, cells were harvested from subconfluent cultures using trypsin and resuspended in fresh medium before plating.

Cells are seeded in 96 well microtiter plates, at 5×10³ cells per well in aliquots of 195 μL medium, and they are allowed to attach to the plate surface by growing in drug free medium for 18 hours. Afterward, samples are added in aliquots of 5 μL in a ranging from 10 to 10⁻⁸ μg/mL, dissolved in DMSO:EtOH:PBS (0.5:0.5:99). After 48 hours exposure, the antitumor effect are measured by the SRB methodology: cells are fixed by adding 50 μL of cold 50% (wt/vol) trichloroacetic acid (TCA) and incubated for 60 minutes at 4° C. Plates are washed with deionised water and dried. 100 μL of SRB solution (0.4% wt/vol in 1% acetic acid) is added to each microtiter well and incubated for 10 minutes at room temperature. Unbound SRB is removed by washing with 1% acetic acid. Plates are air dried and bound stain is solubilized with Tris buffer. Optical densities are read on a automated spectrophotometric plate reader at a single wavelength of 490 nm.

The values for mean+/−SD of data from triplicate wells are calculated. Some parameters for cellular responses can be calculated: GI=growth inhibition, TGI=total growth inhibition (cytostatic effect) and LC=cell killing (cytotoxic effect).

Table 2 illustrates data on the biological activity of the Compound A, B and 19.

TABLE 2
Activity Data (Molar)

| | | Prostate | | Ovary | | Breast | Melanoma |
|---|---|---|---|---|---|---|---|
| | | DU-145 | LN-caP | IGROV | IGROV-ET | SK-BR3 | SK-MEL-28 |
| Compound A | GI50 | 4.24E−7 | 3.33E−7 | 5.79E−7 | 4.63E−7 | 7.89E−7 | 6.49E−7 |
| | TGI | 8.80E−7 | 6.73E−7 | 1.53E−6 | 1.26E−6 | 1.20E−6 | 2.49E−6 |
| | LC50 | 1.83E−6 | 1.37E−6 | 5.31E−6 | 4.42E−6 | 1.81E−6 | 7.66E−6 |
| Compound B | GI50 | >9.29E−6 | — | >9.29E−6 | >9.29E−6 | >9.29E−6 | >9.29E−6 |
| | TGI | >9.29E−6 | — | >9.29E−6 | >9.29E−6 | >9.29E−6 | >9.29E−6 |
| | LC50 | >9.29E−6 | — | >9.29E−6 | >9.29E−6 | >9.29E−6 | >9.29E−6 |

| | | NSCL | Pancreas | Colon | | | Cervix | |
|---|---|---|---|---|---|---|---|---|
| | | A549 | PANC1 | HT29 | LOVO | LOVO-DOX | HELA | HELA-APL |
| Compound A | GI50 | 1.22E−6 | 5.35E−7 | 1.01E−6 | 4.75E−7 | 1.78E−6 | 8.01E−7 | 5.40E−7 |
| | TGI | 3.61E−6 | 1.16E−6 | 2.98E−6 | 8.17E−7 | 5.14E−6 | 2.42E−6 | 1.07E−6 |
| | LC50 | 1.25E−5 | 2.91E−6 | 8.42E−6 | 1.41E−6 | 1.30E−5 | 1.04E−6 | 2.11E−6 |
| Compound B | GI50 | >9.29E−6 | >9.29E−6 | >9.29E−6 | >9.29E−6 | >9.29E−6 | >9.29E−6 | >9.29E−6 |
| | TGI | >9.29E−6 | >9.29E−6 | >9.29E−6 | >9.29E−6 | >9.29E−6 | >9.29E−6 | >9.29E−6 |
| | LC50 | >9.29E−6 | >9.29E−6 | >9.29E−6 | >9.29E−6 | >9.29E−6 | >9.29E−6 | >9.29E−6 |

| | | | Compound 19 |
|---|---|---|---|
| Breast | MDA-MB-231 | GI₅₀ | 6.19E−6 |
| | | TGI | 6.51E−6 |
| | | LC₅₀ | 6.83E−6 |
| Colon | HT29 | GI₅₀ | 2.70E−6 |
| | | TGI | 3.65E−6 |
| | | LC₅₀ | 4.92E−6 |
| NSCL | A549 | GI₅₀ | 5.56E−6 |
| | | TGI | 7.62E−6 |
| | | LC₅₀ | 1.05E−5 |

1. An isolated compound of general formula I: 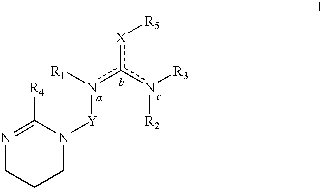
wherein R₁, R₂, R₃ and R₅ are each independently selected from the group consisting of H, OH, NO₂, NH₂, SH, CN, halogen, C(═O)H, CO₂H, COOalkyl, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₄-C₁₈ aryl, substituted or unsubstituted C₄-C₁₈ heterocyclic group, substituted or unsubstituted C₁-C₁₂ alkoxy and substituted or unsubstituted C₂-C₁₂ acyl;
Y is selected from the group consisting of substituted or unsubstituted C₁-C₁₂ alkylene, substituted or unsubstituted C₂-C₁₂ alkenylene and substituted or unsubstituted C₂-C₁₂ alkynylene;
X is selected from the group consisting of O, S and NRₐ;
Rₐ is selected from the group consisting of H, OH, NO₂, NH₂, SH, CN, halogen, C(═O)H, CO₂H, COOalkyl, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₄-C₁₈ aryl, substituted or unsubstituted C₄-C₁₈ heterocyclic group, substituted or unsubstituted C₁-C₁₂ alkoxy and substituted or unsubstituted C₂-C₁₂ acyl;
R₄ is selected from the group consisting of substituted or unsubstituted C₁-C₃₀ alkyl, substituted or unsubstituted C₂-C₃₀ alkenyl, substituted or unsubstituted C₂-C₃₀ alkynyl and substituted or unsubstituted C₄-C₃₀ alkenynyl; and
the dotted line represents an optionally additional bond which is placed in Nₐ—C_{b}, being R₁ absent, in C_{b}—X, being R₅ absent or in C_{b}—N_{c}, being R₂ absent;
wherein the alkyl, alkenyl, alkynyl, alkenynyl, alkylene, alkenylene and alkynylene groups are optionally substituted by a group selected from OH, NO₂, SH, CN, halogen, C(═O)H, optionally substituted C₁-C₁₂ alkoxy, optionally substituted C₁-C₁₂ alkanoyloxy, optionally substituted C₄-C₁₉ aroyloxy, optionally substituted C₄-C₁₆ aralkanoyloxy, halogen, optionally substituted C₄-C₁₈ aryl, amino, mono-(C₁-C₁₂ alkyl)amino and di-(C₁-C₁₂ alkyl)amino, optionally substituted guanidine, optionally substituted C₁-C₁₂ alkoxycarbonyl, optionally substituted C₄-C₁₁ aryloxycarbonyl, optionally substituted C₄-C₁₁ aralkyloxycarbonyl, carbamoyl, N—(C₁-C₂₀ alkyl)carbamoyl and N,N-di-(C₁-C₂₀ alkyl)carbamoyl;
or a pharmaceutically acceptable salt, tautomer, derivative, prodrug or stereoisomer thereof.

2. The compound according to claim 1, having the following formula II: 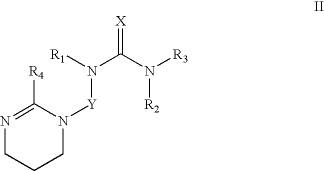
wherein R₁, R₂, R₃, R₄ and Y are as defined in claim 1, and X is selected from the group consisting of O, S and NH.

3. The compound according to claim 1 wherein Y is a substituted or unsubstituted C₁-C₆ alkylene and X is NH.

4. The compound according to claim 1, wherein R₁, R₂, R₃ and R₅ are each independently selected from the group consisting of H, OH, NO₂, NH₂, SH, CN, halogen, C(═O)H, CO₂H, COOalkyl, substituted or unsubstituted C₁-C₆ alkyl and substituted or unsubstituted C₂-C₆ acyl.

5. The compound according to claim 2, wherein R₁, R₂ and R₃ are each independently selected from the group consisting of H, OH, NO₂, NH₂, SH, CN, halogen, C(═O)H, CO₂H, substituted or unsubstituted C₁-C₆ alkyl and substituted or unsubstituted C₂-C₆ acyl.

6. The compound according to claim 1, wherein R₄ is: 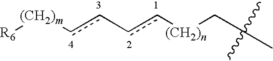
wherein n is an integer from 1 to 12;
m is an integer from 1 to 10;
R₆ is selected from the group consisting of H, OH, NO₂, SH, CN, halogen, C(═O)H, optionally substituted C₁-C₁₂ alkoxy, optionally substituted C₁-C₁₂ alkanoyloxy, optionally substituted C₄-C₁₈ aroyloxy, optionally substituted C₄-C₁₆ aralkanoyloxy, optionally substituted C₄-C₁₈ aryl, amino, mono-(C₁-C₁₂ alkylamino, di-(C₁-C₁₂ alkylamino, optionally substituted guanidine, optionally substituted C₁-C₁₂ alkoxycarbonyl, optionally substituted C₄-C₁₁ aryloxycarbonyl, optionally substituted C4-C₁₁ aralkyloxycarbonyl, carbamoyl, N—(C₁-C₂₀ alkyl)carbamoyl and N,N-di-(C₁-C₂₀ alkyl)carbamoyl;
and the dotted line represents an optional additional single or double bond.

7. The compound according to claim 6, wherein n is an integer from 1 to 8, m is an integer from 1 to 5 and there is a double bond placed between C₁-C₂ and a triple bond placed between C₃-C₄.

8. The compound according to claim 1, having the following formula 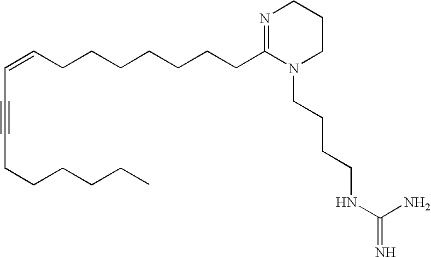
or a pharmaceutically acceptable salt, tautomer, derivative, prodrug or stereoisomer thereof.

9. The compound according to claim 1, having the following formula 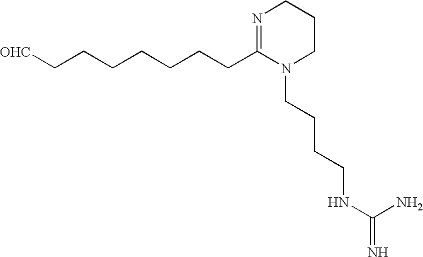
or a pharmaceutically acceptable salt, tautomer, derivative, prodrug or stereoisomer thereof.

10. The compound according to claim 1, having the following formula 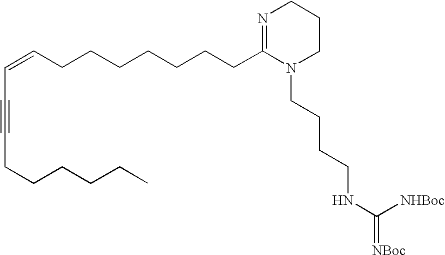
or a pharmaceutically acceptable salt, tautomer, derivative, prodrug or stereoisomer thereof.

11. The compound according to claim 1, wherein the compound is in the form of its trifluoroacetate salt.

12. A process for obtaining a compound as defined in claim 1, which comprises an extraction and isolation from the coral Meandrina meandrites.

13. A pharmaceutical composition comprising a compound according to claim 1, or a pharmaceutically acceptable salt, tautomer, derivative, prodrug or stereoisomer thereof, and a pharmaceutically acceptable diluent or carrier.

14. A method of making a medicament comprising isolating a compound according to claim 1 or a pharmaceutically acceptable salt, tautomer, derivative, prodrug or stereoisomer thereof, and combining said compound with a pharmaceutically acceptable diluent or carrier.

15. The method according to claim 14, wherein the medicament is for the treatment of cancer.

16. A method of treating cancer comprising administering an effective amount of a compound according to claim 1 to a patient in need thereof.

17. A method of killing or inhibiting the growth of cancer cells comprising administering a compound according to claim 1 to cancer cells.
